(19) United States
(12) Patent Application Publication
(10) Pub. No.: US 2021/0174906 A1
(43) Pub. Date: Jun. 10, 2021

(54) Systems And Methods For Prioritizing The Selection Of Targeted Genes Associated With Diseases For Drug Discovery Based On Human Data

(71) Applicant: ACCENTURE GLOBAL SOLUTIONS LIMITED, Dublin (IE)

(72) Inventors: Qurrat UL AIN, Dublin (IE); Mykhaylo ZAYATS, Dublin (IE); Patrick MOREAU, Dublin (IE); Fiona BRENNAN, Dublin (IE); Sumit PAI, Dublin (IE); Luca COSTABELLO, Newbridge (IE); Sean GORMAN, Goatstown (IE)

(21) Appl. No.: 16/818,412

(22) Filed: Mar. 13, 2020

Related U.S. Application Data
(60) Provisional application No. 62/944,769, filed on Dec. 6, 2019.
(60) Provisional application No. 62/954,901, filed on Dec. 30, 2019.

(51) Int. Cl. G16B 40/00; G16B 25/00; G16B 50/30; G06F 17/18; G16B 5/00

(52) U.S. Cl. CPC G16B 40/00; G16B 25/00; G16B 5/00; G06F 17/18; G16B 50/30

(57) ABSTRACT

Systems and methods enable the discovery of new relationships between diseases and genes by prioritizing the selection of gene targets for a disease using an embedding space generated from a knowledge graph by mapping datasets collected from various data sources using a graph schema, modeling disease and gene associations with link weightings, analyzing the data with several machine learning models, and scoring predictions.

### CROSS-REFERENCE TO RELATED APPLICATION

This application claims benefit to U.S. Provisional patent Application No. 62/944,769 filed on Dec. 6, 2019, and U.S. Provisional patent Application No. 62/954,901 filed on Dec. 30, 2019, the entireties of which are incorporated by reference herein.

### FIELD OF THE INVENTION

The present disclosure relates in general to the fields of target identification and validation using human genetics data and human disease ontology, and in particular methods and systems for discovering new relationships between diseases and genes by prioritizing the selection of targeted genes associated with a disease.

### BACKGROUND

Basic techniques and equipment for machine learning, modeling data, graph embedding, and ranking drug compounds based on experimental data are known in the art. Enterprise systems have access to large volumes of information, both proprietary and public, relating to human genetic makeup, genetic mutation information, gene expression information, drug interactions, molecular structures, and disease classification. Existing analytical applications and data warehousing systems have not been able to fully utilize such information. Often times, information is simply aggregated into large data warehouses without proper data quality screening and the inclusion of an added layer of relationship data connecting the information. Such aggregation of large amounts of data, without contextual or relational information, are data dumps that are not useful.

Information stored in data warehouses are likely to be stored in their original format, thus expending large amounts of computing resources to transform the information into searchable data in order to respond to a query. Traditional approaches for searching enterprise data typically entail using string matching mechanisms (semantic linking) without context. However, such previous approaches are limited in their ability to provide queried data. Moreover, most of the stored data is not easily searchable or available for machine learning analytics. Accordingly, conventional knowledge query systems return results that do not provide a complete picture of knowledge and data available in the enterprise. A multi-relational link prediction is desired to more efficiently and effectively identify gene targets for diseases.

### SUMMARY

The present disclosure describes a system for identifying a gene (target) associated with a disease. The system includes a memory to store executable instructions; and a processor adapted to access the memory. The processor is further adapted to execute the executable instructions stored in the memory to extract datasets from a plurality of databases, the extracted datasets comprising historical datasets for a genetic mutation in a human DNA dataset, and/or a gene expression dataset, and/or a gene interaction dataset, and/or a drug dataset, and/or a disease dataset. The instructions when executed store the extracted datasets in a data lake. The data lake is stored in the memory in graph-based datasets that include a subject, an object and a predicate. The instructions when executed generate a knowledge graph based on the data lake, with the knowledge graph representing a plurality of links related to at least one gene and at least one disease.

The present disclosure also describes a method for identifying a target gene associated with a disease. The method includes extracting, by a device, datasets from a plurality of databases, the extracted datasets comprising historical datasets for a genetic mutation in a human DNA dataset, and/or a gene expression dataset, and/or a gene interaction dataset, and/or a drug dataset, and/or a disease dataset. The device includes a memory and a processor in communication with the memory. The method includes storing, by the device, the extracted datasets in a data lake. The data lake is stored in the memory in graph-based datasets that include a subject, an object and a predicate. The method generates, by the device, a knowledge graph based on the data lake, with the knowledge graph representing a plurality of links related to at least one gene and at least one disease.

The present disclosure also describes a non-transitory computer-readable medium including instructions configured to be executed by a processor. The executed instructions are adapted to cause the processor to extract datasets from a plurality of databases, the extracted datasets comprising historical datasets for a genetic mutation in a human DNA dataset, and/or a gene expression dataset, and/or a gene interaction dataset, and/or a drug dataset, and/or a disease dataset. The instructions are configured to store the extracted datasets in a data lake, where the data lake is stored in a memory in communication with the processor. The data lake is stored in graph-based datasets, with each of the graph-based datasets including a subject, an object and a predicate. The instructions are configured to generate a knowledge graph based on the data lake, with the knowledge graph representing a plurality of links related to at least one gene and at least one disease.

### BRIEF DESCRIPTION OF THE DRAWINGS

The foregoing and other objects, features, and advantages for embodiments of the present disclosure will be apparent from the following more particular description of the embodiments as illustrated in the accompanying drawings, in which reference characters refer to the same parts throughout the various views. The drawings are not necessarily to scale, emphasis instead being placed upon illustrating principles of the present disclosure.

FIG. 1 is a flow diagram illustrating an example of a method implemented by an exemplary system, in accordance with certain embodiments of the present disclosure.

FIG. 2 is a block diagram illustrating an example of an architecture for an exemplary system, in accordance with certain embodiments of the present disclosure.

FIG. 3 is a block diagram illustrating an example of an architecture for the data mapping that may be implemented by the system, in accordance with certain embodiments of the present disclosure.

FIG. 4 is a schematic diagram illustrating a knowledge graph schema, in accordance with certain embodiments of the present disclosure.

FIGS. 5(a)-(d) illustrate such unified visualizations, in accordance with certain embodiments of the present disclosure.

FIGS. 6(a)-(b) illustrates diagrams for the weight generation pipelines, in accordance with certain embodiments of the present disclosure.

FIGS. 7(a)-(b) illustrate a network graph and a screenshot representation of an exemplary KnowGene approach for such a graph, in accordance with certain embodiments of the present disclosure.

FIG. 8 is a schematic diagram an exemplary non-weighted AmpliGraph model, in accordance with certain embodiments of the present disclosure.

FIGS. 9(a)-(b) illustrate such a graph and genetic information for an exemplary R-GCN model, in accordance with certain embodiments of the present disclosure.

FIG. 10 illustrates a graphical representation of an exemplary function association score, in accordance with certain embodiments of the present disclosure.

FIG. 11 illustrates an exemplary density graph representing the distance to the core genes, in accordance with certain embodiments of the present disclosure.

FIG. 12 illustrates an exemplary density graph representing the distance to the disease genes, in accordance with certain embodiments of the present disclosure.

FIG. 13 is a block diagram illustrating an embodiment of a system, in accordance with certain embodiments of the present disclosure.

FIG. 14 is a block diagram illustrating an embodiment of a computer architecture for a computer device for implementing the exemplary system shown in FIG. 13, in accordance with certain embodiments of the present disclosure.

### DETAILED DESCRIPTION

Reference will now be made in detail to the embodiments of the present disclosure, examples of which are illustrated in the accompanying drawings, which form a part of the present disclosure, and which show, by way of illustration, specific examples of embodiments. Please note that the disclosure may, however, be embodied in a variety of different forms and therefore, the covered or claimed subject matter is intended to be construed as not being limited to any of the embodiments to be set forth below. Please also note that the disclosure may be embodied as methods, devices, components, or systems. Accordingly, embodiments of the disclosure may, for example, take the form of hardware, software, application program interface (API), firmware or any combination thereof.

Throughout the specification and claims, terms may have nuanced meanings suggested or implied in context beyond an explicitly stated meaning. Likewise, the phrase “in one embodiment” or “in one implementation” as used herein does not necessarily refer to the same embodiment or implementation and the phrase “in another embodiment” or “in another implementation” as used herein does not necessarily refer to a different embodiment or implementation. It is intended, for example, that claimed subject matter includes combinations of exemplary embodiments or implementations in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” or “at least one” as used herein, depending at least in part upon context, may be used to describe any feature, structure, or characteristic in a singular sense or may be used to describe combinations of features, structures or characteristics in a plural sense. Similarly, terms, such as “a”, “an”, or “the”, again, may be understood to convey a singular usage or to convey a plural usage, depending at least in part upon context. In addition, the term “based on” or “determined by” may be understood as not necessarily intended to convey an exclusive set of factors and may, instead, allow for existence of additional factors not necessarily expressly described, again, depending at least in part on context.

The present disclosure may be embodied in various forms, including a system, a method, a computer readable medium, or a platform-as-a-service (PaaS) product for prioritizing the selection of targeted genes associated with diseases based on human data. In certain embodiments, the most informed gene targets for a disease may be identified based on human biological data. In an example, the present disclosure may be applied to drug discovery for diseases such as immunology, inflammatory bowel disease (IBD), rheumatoid arthritis (RA) and neurodegeneration.

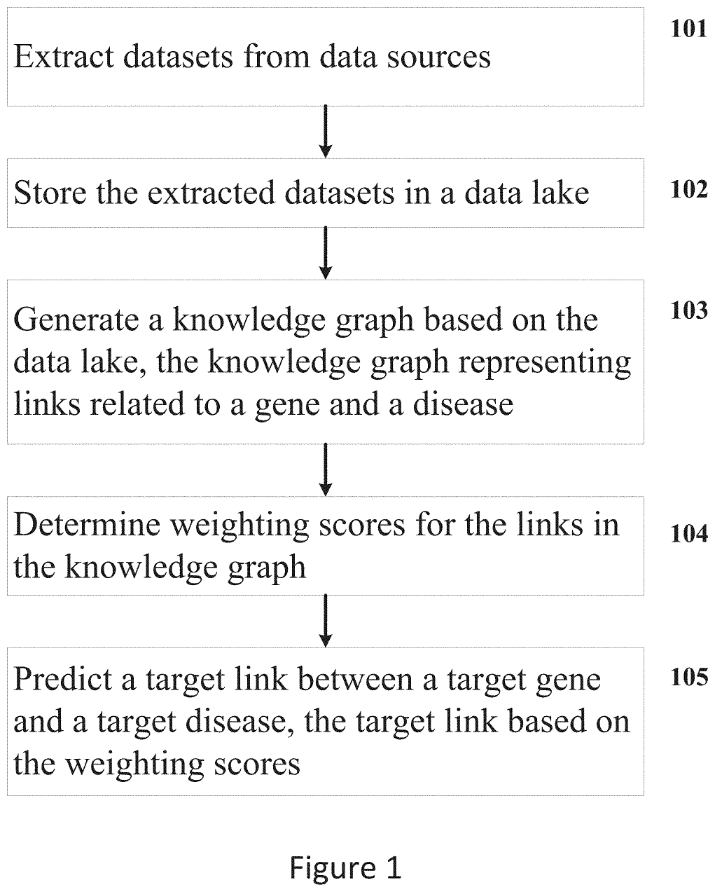

In certain embodiments, as illustrated in FIG. 1, a method or system for identifying a gene (target) associated with a disease may include the steps or functionality of: extracting human biological datasets from databases or data sources (block 101); storing the extracted datasets in a data lake (block 102); generating a knowledge graph based on the data lake, wherein the knowledge graph represents associations or links related to a gene and a disease (block 103); determining weighting scores for the links in the knowledge graph (block 104); and predicting a target link between a target gene and a target disease based on the weighting scores (block 105). The method and/or system may further include the displaying or visualization of the knowledge graph, and/or the predicted genes and its links.

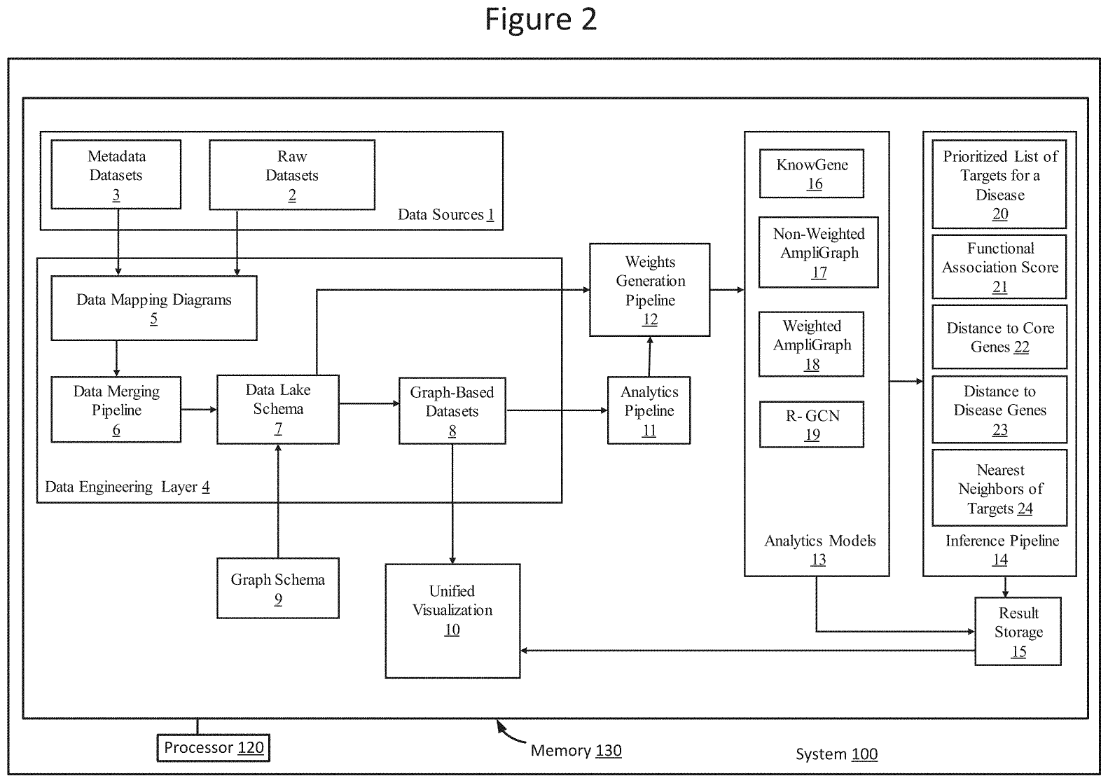

FIG. 2 illustrates an embodiment of such a system 100 that may be implemented in many different ways, using various components and modules, including any combination of circuitry described herein, such as hardware, software, middleware, application program interfaces (APIs), and/or other components for implementing the features of the circuitry such as a processor 120 and memory 130. The system 100 may include, for example: data sources 1 including raw datasets 2 and metadata datasets 3; a data engineering layer 4 including data mapping diagrams 5, a data merging pipeline 6, a data lake schema 7, and graph-based datasets 8; a graph schema 9; an unified visualization 10; an analytics pipeline 11; a weights generation pipeline 12; analytics models 13; an inference pipeline 14; and a result storage 15. The analytics models 13 may include may include: a knowledge-based model for predicting gene-disease associations, such as the KnowGene model 16; a non-weighed model, such as the Non-Weighted AmpliGraph model 17; a weighted model, such as the Weighted AmpliGraph model 18; and a Relational Data with Graph Convolutional Networks (R-GCN) model 19. The inference pipeline 14 may include: a prioritized list 20 of targeted genes for a disease, a functional association score 21, a first distance 22 between a targeted gene and core genes, a second distance 23 between a targeted gene and disease genes, and nearest neighbors 24 of targeted genes.

In some embodiments, the data sources 1 may include numerous raw data sources and numerous metadata sources. Metadata datasets 3 may be extracted from the metadata sources, and raw datasets 2 may extracted from the raw data sources. The metadata datasets 3 may be configured to map the raw datasets 2 received from the raw data sources. For example, one raw dataset 2 may reference the disease commonly known as diabetes, while another raw dataset 2 may refer to the same disease by using its formal name, diabetes mellitus. The extracted metadata datasets 3 may associate key identifiers from the two raw datasets 2. In addition, the extracted metadata datasets 3 may provide information to facilitate annotation of the extracted raw datasets 2. As an application ontology, the metadata datasets 3 may be configured to allow other reference ontologies to be mapped to it, and may enable the determination of broader relationships. For example, the ChEMBL, Ensembl and EFO metadata sources may be utilized in the data mapping process, in accordance with embodiments of the present disclosure. The ChEMBL is a data source 1 that may provide information about known drugs. The Ensembl or EQTL data source 1 may provide information relating to associations between gene identifier and gene labels. EFO metadata datasets 3 may include information relating to disease anthologies, which may be used for annotations and/or mapping datasets received from raw data sources 2. Raw data sources may be utilized within the training process. In certain embodiments, the following raw data sources may be implemented: genome-wide association study (GWAS), SpringDB, GeneAtlas, Genotype-Tissue Expression (GTEX), NealeLab, and/or PheWeb.

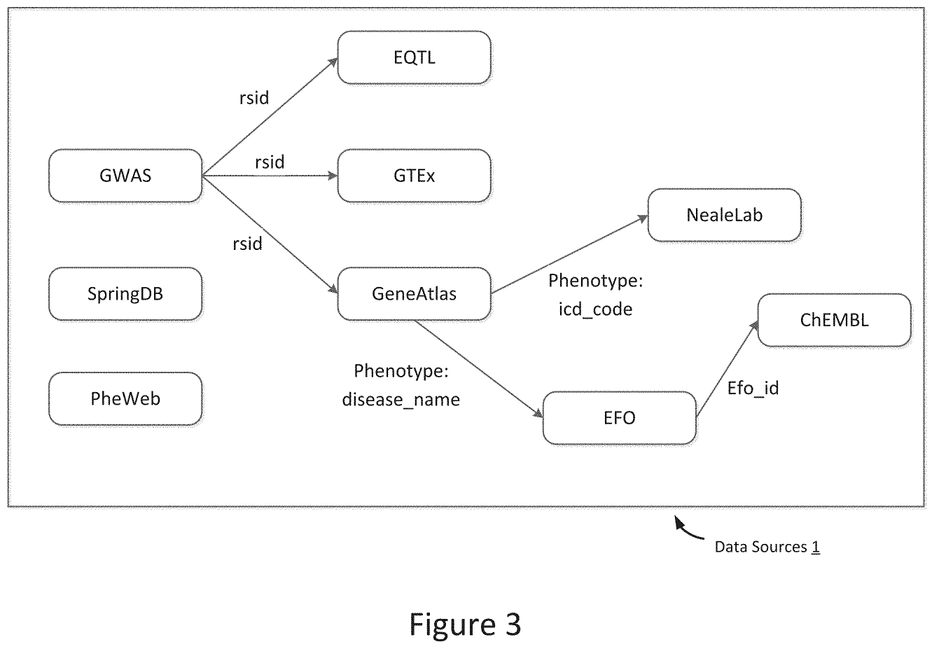

FIG. 3 illustrates an example of an architecture or diagram for a data mapping implemented by the system 100, in accordance with certain embodiments of the present disclosure. As shown in FIG. 3, the data sources 1 may include the six raw data sources and the three metadata sources described above. An example of a key identifier includes the reference single nucleotide polymorphism (SNP) identifier, known as rsid.

The system 100 may include a data mapping process for generating a catalogue where the phenotype ontologies found across various data sources are mapped to one Standard Disease Ontology (SDO). Accordingly, the system 100 may maintain key mappings between the various data sources 1 that are used for merging the raw datasets 2 together. The data mapping process may utilize the metadata datasets 3 to link and merge the raw datasets 2.

Accordingly, the system 100 may be adapted to combine information received from a diverse group of public data sources 1. Such diverse data sources 1 may include heterogeneous datasets. For example, some the datasets extracted by the system 100 may comprise text, while other extracted datasets may be numerical in nature. In an embodiment, the datasets may include pathway information, genetic profiles and disease anthologies. The system 100 may integrate the different genetic datasets and disease anthologies based on the data mapping diagrams 5 shown in FIG. 2, merge the datasets, and apply analytical machine learning (ML) methods to identify and prioritize the targeted genes against a disease. The mapping of the diverse knowledge base received from various data sources 1 may be based on the unique identifiers that link the raw datasets 2. The raw datasets 2 and metadata datasets 3 received from the data sources 1 may be utilized to define the knowledge base used to generated a knowledge graph via the data engineering layer 4.

As illustrated in FIG. 2, the data engineering layer 4 may include data mapping diagrams that define standard entity relations and contact one received dataset with another receive dataset based on a primary key that joins the two datasets together. The data mapping diagrams may be further utilized to merge the raw datasets 2 together via the data merging pipeline 6. In certain embodiments, the data merging pipeline 6 of the system 100 may download datasets in different formats from the data sources 1. Further, the data merging pipeline 6 may process or clean the received datasets 2/3 to configure them into a format that may be merged together using key mappings from a catalogue. The system 100 may merge the received datasets based on the data mapping diagrams used to generate the mapped datasets. The system 100 may store of the merged datasets in a data lake. In its unified format, the datasets stored in the data lake may be configured to be queried for further processing.

In certain embodiments, a data lake schema 7 may be implemented by the data engineering layer 4. Based on the data lake schema 7, the data stored in the data lake may have a natural or raw format. The data lake may include a voluminous repository of datasets including the raw copies of received datasets, as well as the processed or transformed datasets that may be used for the reporting, visualization, advanced analytics and machine learning performed by the system 100. A data lake may store various datasets, including object blobs, structured data from relational databases (e.g., datasets having rows and columns), semi-structured data (e.g., CSV, logs, XML, JSON), unstructured data (e.g., documents, PDFs) and binary data (e.g., images, audio, video). In some embodiments, the data lake may contain only the merged datasets discussed above. As such, the datasets 2/3 received from the data sources 1 may be combined together in an unified format, mapped using primary keys, and stored on a data lake. This datasets may be the source of the analytics pipeline, and may be used for visualization and further analysis in the analytics pipeline 11.

The data engineering layer 4 may include the step of generating graph-based datasets (e.g., triples) that may include the datasets 2/3 received from each data sources 1. In certain embodiments, the processed datasets may be configured in columns that include the subjects and objects for triples, and the predicates that tie the two subjects and objects together. In some embodiments, this step must be conducted for every new dataset. This step may result in the graph-based datasets, such as triples, that may be used in the analytics models 13. As shown in FIG. 2, the analytics models 13 may include a knowledge-based model 16 for predicting gene-disease associations (e.g., a KnowGene model); a non-weighed model 17 (e.g., a Non-Weighted AmpliGraph model); a weighted model 18 (e.g., a Weighted AmpliGraph model); and a graph convolutional network model 19 (e.g., a relational data with graph convolutional networks (R-GCN) model).

Further, the data engineering layer 4 may receive input from a graph schema 9, which may represent a blueprint for the knowledge graph. In certain embodiments, the graph schema 9 may define the manner that the received datasets 2/3 are mapped. In some embodiments, the graph schema 9 may be adapted to enable the data merging pipeline 6 to merge predetermined information or features from the received datasets 2/3. Such predetermined information or features to be merged may be based on known relationships that link a gene variant to a gene, or that link a gene to a disease. In an embodiment, the known relationships may include a basis for the association between the gene, its variant, and a disease. The known relationships may be based on information stored in the received datasets 2/3, or additional information received from professionals, practitioners, and scientists in the field of genetics and diseases.

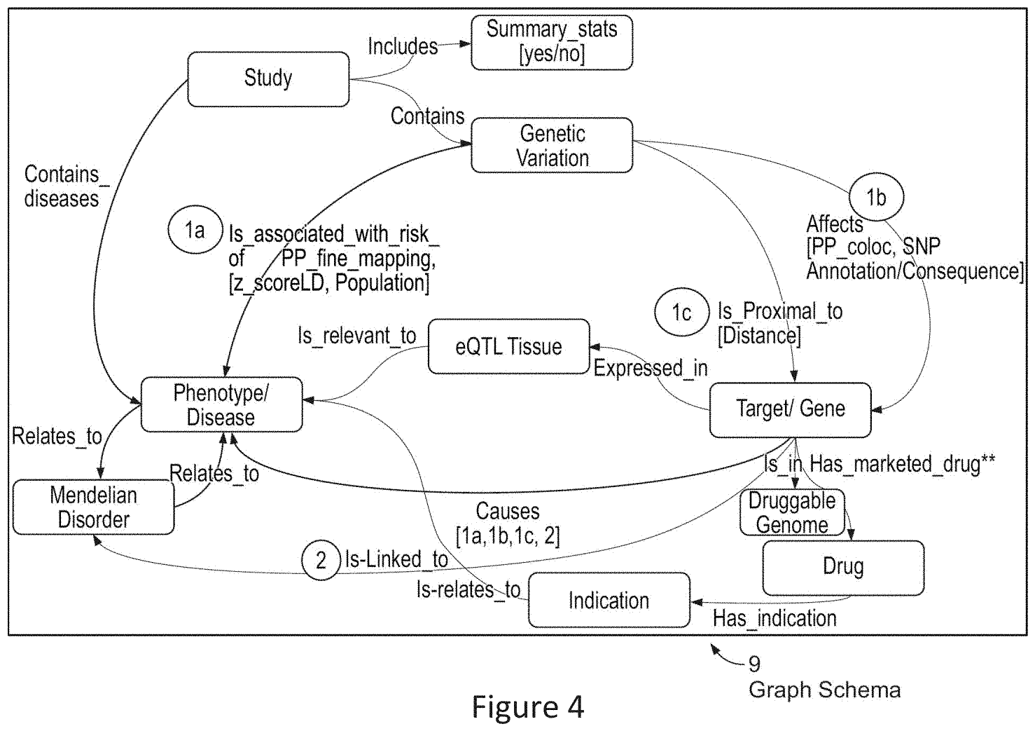

The graph schema 9 may include definitions for the entities, concepts and data used in the analytics models 13. The data lake schema 7 may be based on the graph schema 9. FIG. 4 illustrates a knowledge graph schema 9 that include information pertaining to target genes and their known diseases. Each node may represent a fact or concept relating to genes or diseases, and each edge may represent a relationship between the concepts represented by the two nodes.

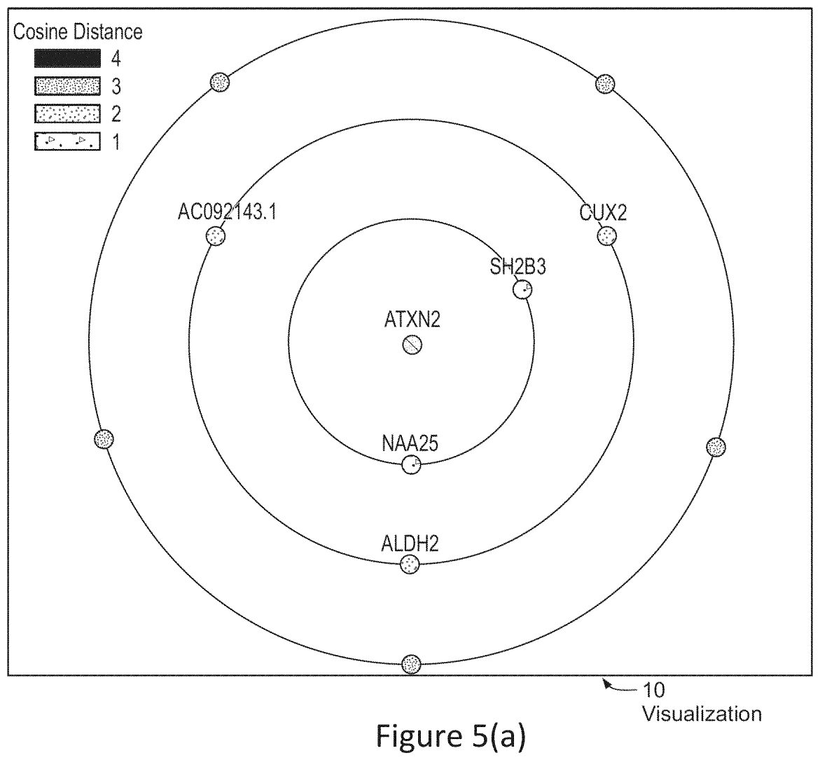

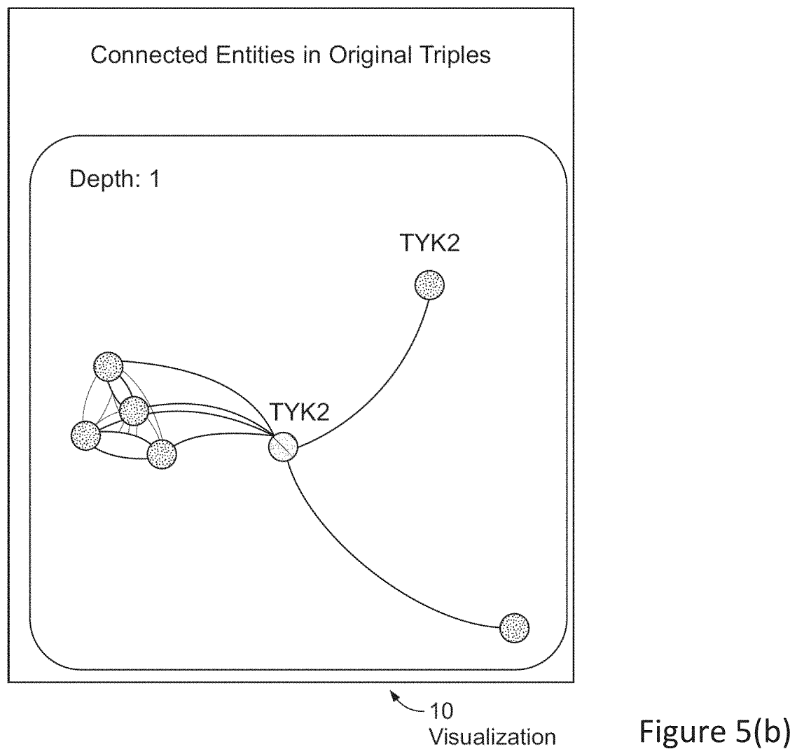

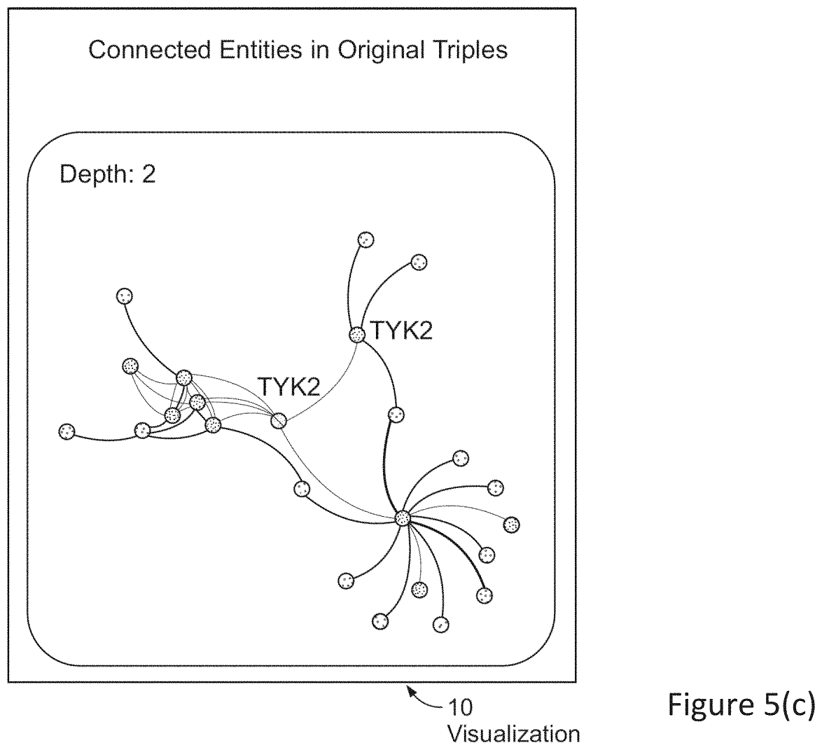

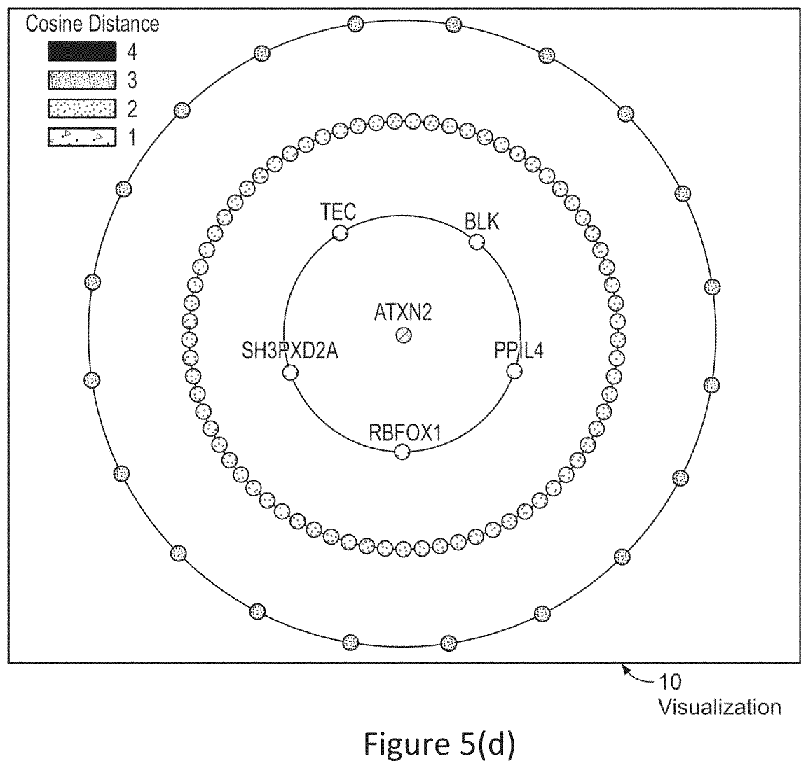

In an embodiment, the unified visualization 10 may include a graphical user interfaces (GUIs). The visualization functionality or step may include, or visually represent, the rationale showing why a certain gene target may be ranked highly. Visualizations 10 may be generated on a distance of targeted gene to ‘core genes’ or ‘nearest neighbours’ in an embeddings space generated based on a knowledge graph via the data engineering layer 4, and may illustrate the connected entities in the knowledge graph. FIGS. 5(a)-(d) illustrate such visualizations 10. Referring back to FIG. 2, the inference pipeline 14 may include a prioritized list 20 of targets for a disease, a functional association score 21, a first distance 22 between a targeted gene and core genes, a second distance 23 between a targeted gene and disease genes, and nearest neighbors 24 of targeted genes. The results from the inference pipeline 14 may also be displayed via visualizations 10.

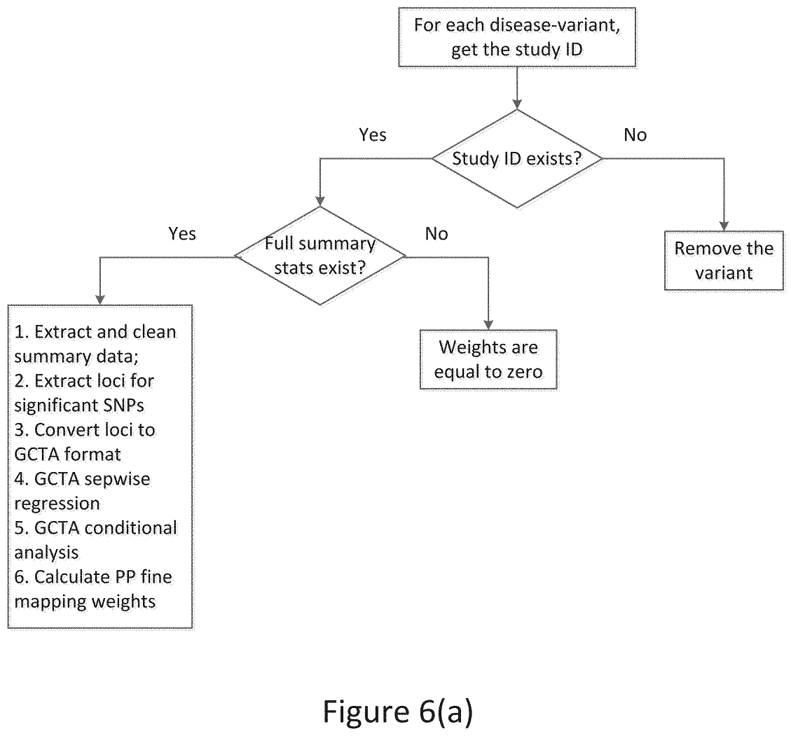

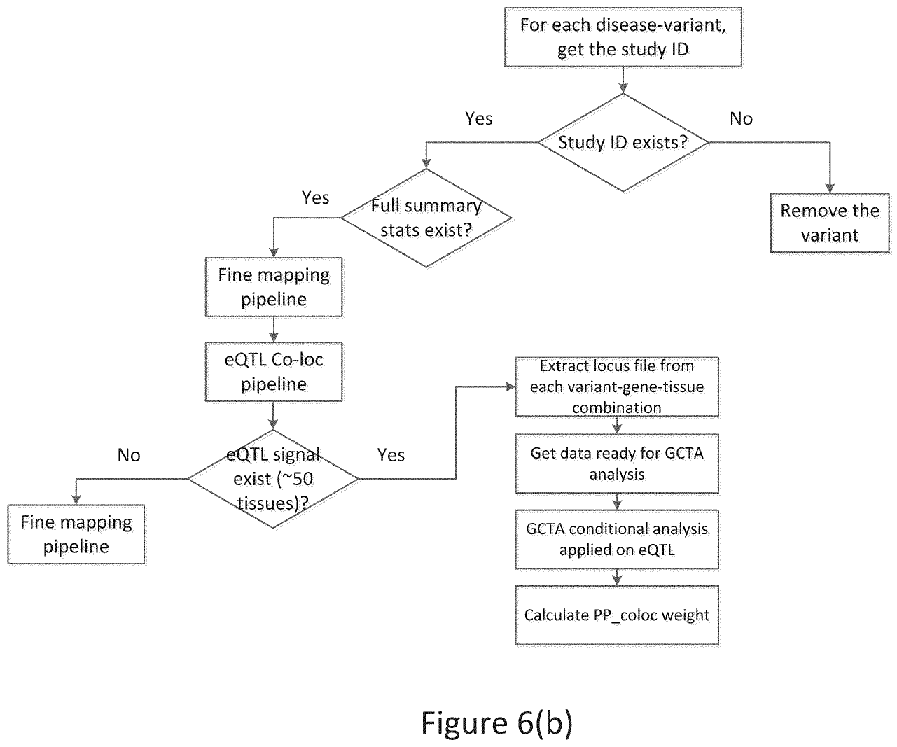

In an embodiment, the weights generation pipeline 12 may generate weights for the links that define the gene-disease associations, which may receive input from the analytics models 13. In some embodiments, the weights may define the importance of the connection between a targeted gene and its associated disease. The weights may define, or represent, whether a gene is important to the existence of a disease. The weights may be compared to determine whether a targeted gene is less or more important to another disease. In an embodiment, the weights may be assigned to disease-to-variant associations and gene-to-variant associations. Such weights may be derived from raw datasets 2 received from the GWAS and GTEx data sources. Methods for generating weights based on such raw datasets 2 and high-level flow diagrams for such weights generation pipelines are shown in FIGS. 6(a)-(b).

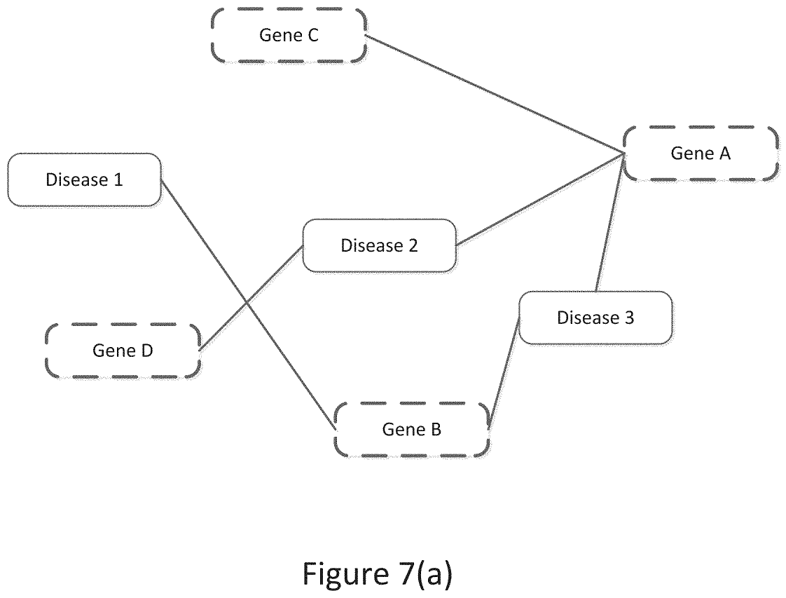

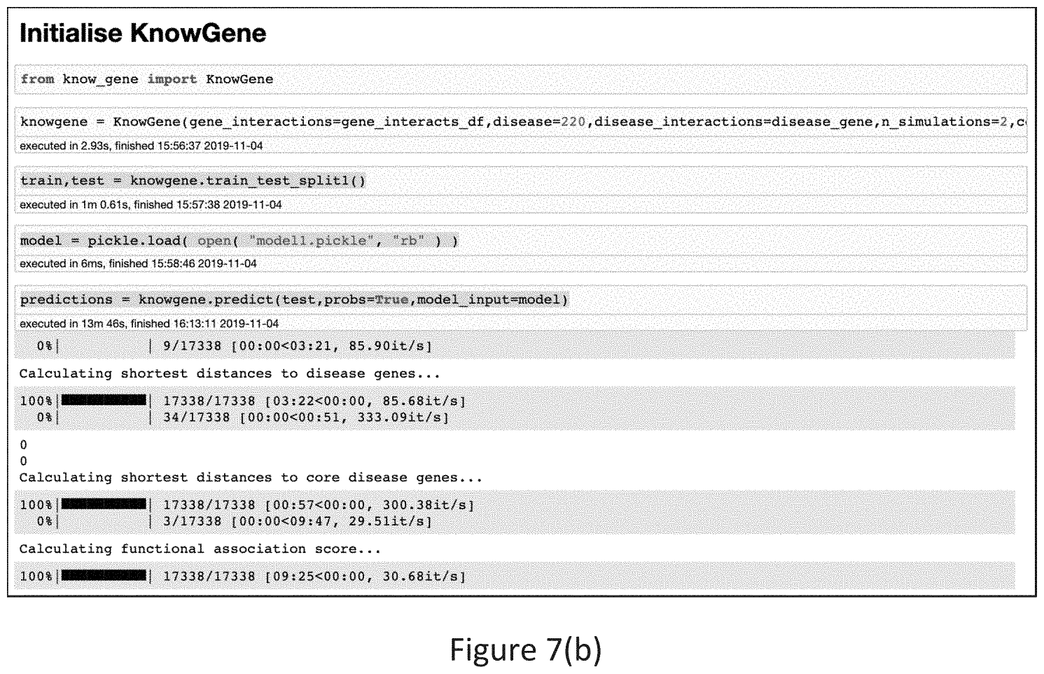

The knowledge-based model 16 for predicting gene-disease associations (e.g., a KnowGene model) may be a machine learning approach to the target identification process. It often utilizes gene-gene data and gene-disease relation data from GWAS and the Online Mendelian Inheritance in Man (OMIM) data source 1 in order to predict gene targets that are associated with a given disease. The KnowGene model 16 may provide a benchmark for the system 100 to compare with the novel analytics approaches presently disclosed. FIGS. 7(a)-(b) illustrate a knowledge graph and a screenshot representation of the initialization and calculations from an exemplary KnowGene model 16 for such a graph.

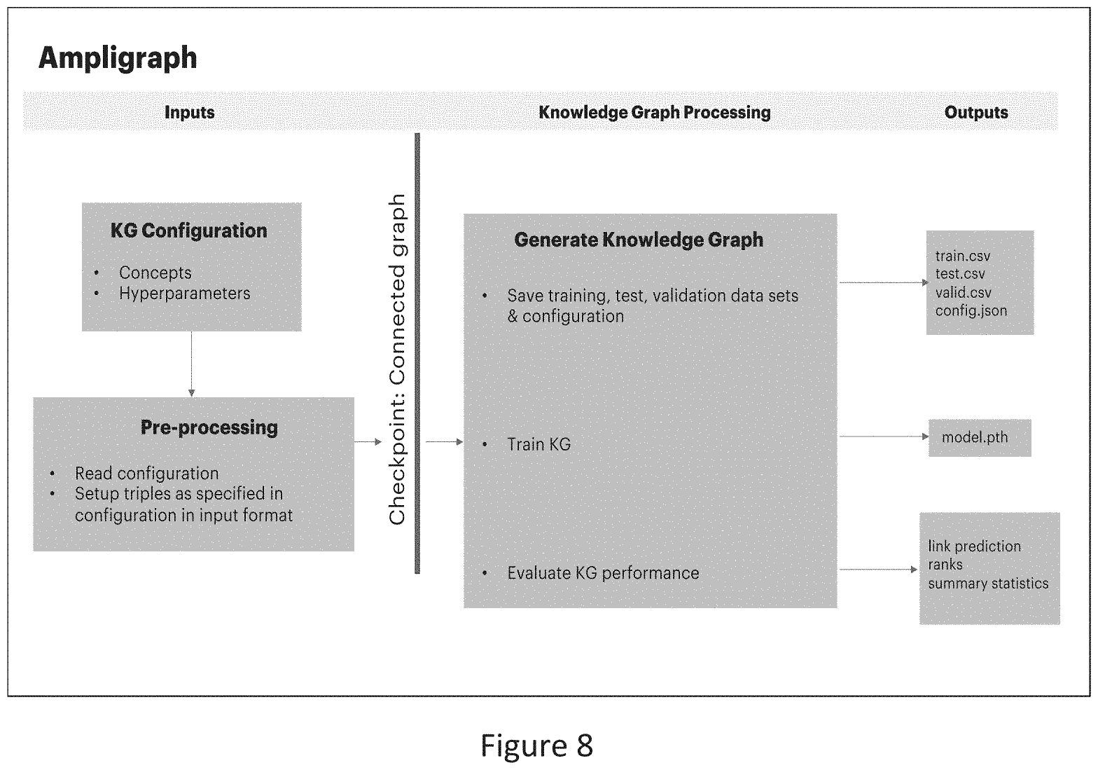

Knowledge Graphs may include graph-based knowledge bases having facts modelled as relationships between entities. Upon the graph, a neural architecture may be built to create embeddings of complex entities. From these embeddings, scoring functions may perform tasks, such as link prediction. In accordance with certain embodiments, this logic may be implemented to discover new relations between diseases and genes. FIG. 8 illustrates an exemplary non-weighted model, such as the Non-Weighted AmpliGraph model 17. As an extension of such an analytical approach, a weighted model may also be implemented, such as the Weighted AmpliGraph model 18. In one implementation, a Weighted AmpliGraph model 18 may be the system as described in the U.S. Provisional patent Application No. 62/954,901 filed on Dec. 30, 2019, the entirety of which is incorporated by reference herein. A difference between such approaches includes additional information relating to the links between entities in the graph. Such information may be used during training. This information may be incorporated in order to update the embeddings of each entity to improve the accuracy of the predictions.

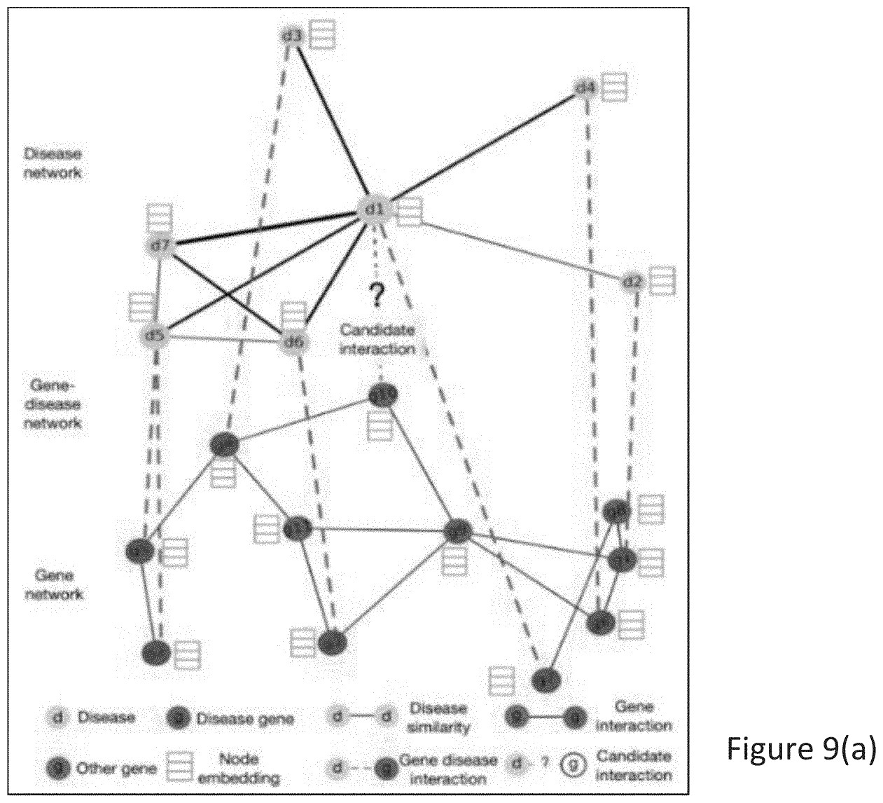

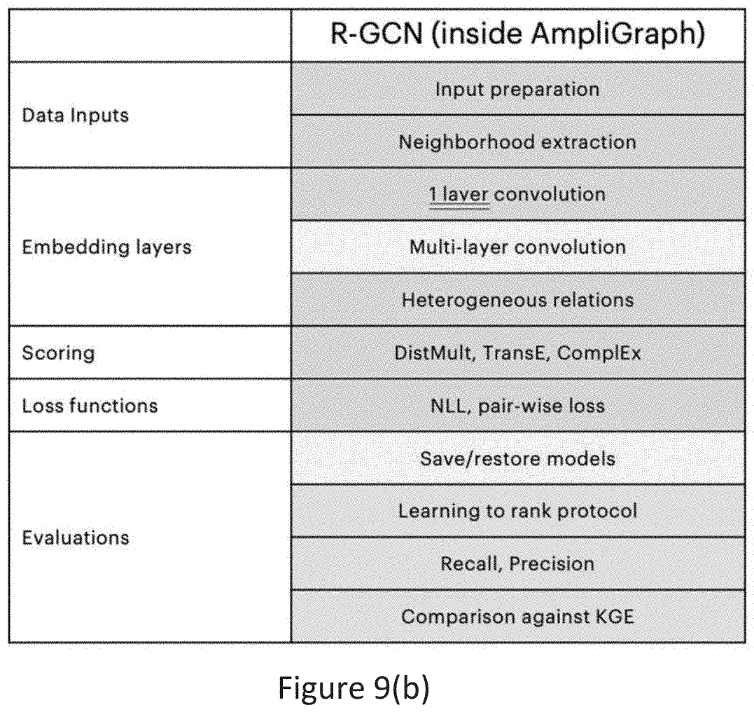

The graph convolutional network model 19 (e.g., a R-GCN model) may include a machine learning approach for building ontologies based on a graph structure. The R-GCN model 19 may be used to represent genetic information in a graph, and to discover new relations between diseases and genes. FIGS. 9(a)-(b) illustrate such a graph and genetic information for an exemplary R-GCN model 19.

In some embodiments, each model may require a test set of verified, validated gene targets so that the performance of the models may be evaluated. This may comprise a prioritized list 20 of gene targets for a disease. In an example concerning a set of validated targets for rheumatoid arthritis, the analytics models 13 may be assigned with the task of predicting targets for rheumatoid arthritis. The validated dataset may be used with a binary classification, and/or learn-to-rank metrics, in order to measure each model's performance.

The Functional Association Score 21 may be utilized by the KnowGene model 16. Specifically, this metric may consider the co-occurrences of a query gene with known disease genes. In an embodiment, it may compare the joint probability of the query gene and the disease gene occurring together in a disease against the probability of them occurring independently in a disease. The functional association score 21 may be defined as follows:

$$S ( D , {g}_{x} ) = {Σ}_{{g}_{y} ∈ D} P ( {g}_{x} , {g}_{y} ) I ( {g}_{x} , {g}_{y} ) , \\ where$$

$$I ( {g}_{x} , {g}_{y} ) = log \frac{P ( {g}_{x} , {g}_{y} )}{P ( {g}_{x} ) P ( {g}_{y} )} ,$$

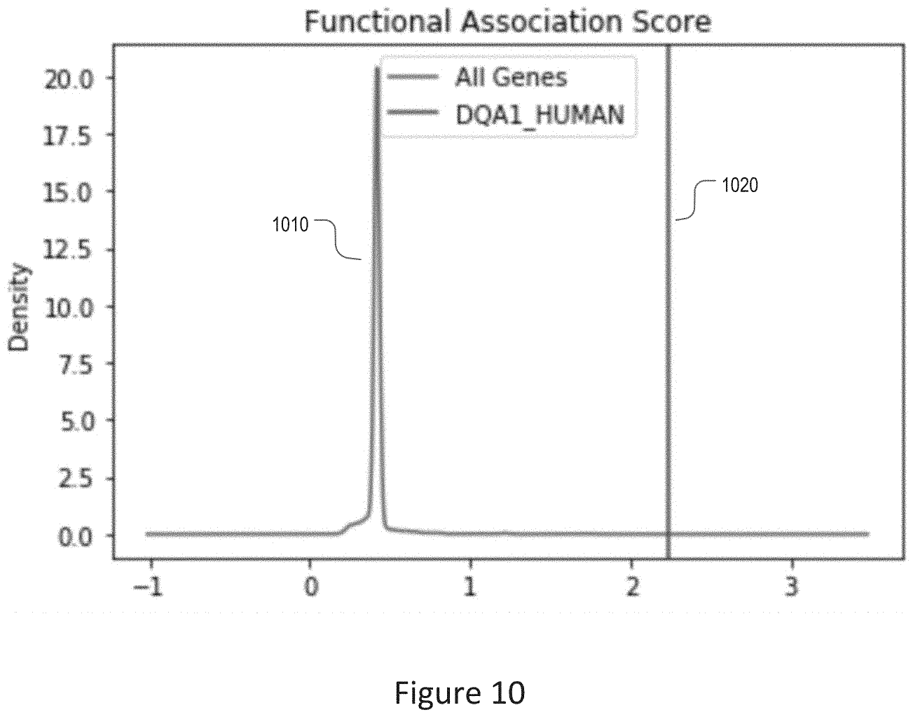

and where P(gₓ), P(g_{y}) are the probabilities of observing genes gₓ and g_{y}, independently in a given disease, and P(gₓ, g_{y}) is the probability of observing genes gₓ and g_{y}, together in a given disease D. FIG. 10 illustrates a graphical representation of an exemplary function association score 21. In FIG. 10, a density curve 1010 is for all genes, and a density curve 1020 is for a gene of DQA1_HUMAN.

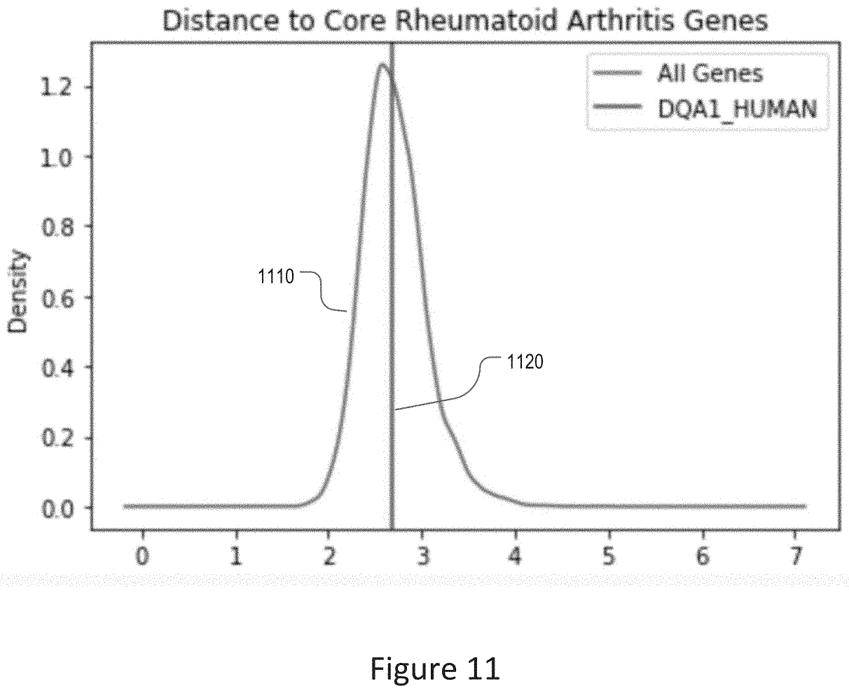

The distance 22 between a targeted gene and core genes may also be utilized by the KnowGene model 16. Core genes may include the genes in the largest statistically significant connected cluster in the interactome, the gene-gene interaction network. Statistical testing may be conducted to ensure that the largest connected cluster is not just randomly formed. FIG. 11 illustrates an exemplary distribution representing the distance 22 to the core genes. If no core genes exist, the histogram values may equal zero. In FIG. 11, a density curve 1110 is for all genes, and a density curve 1120 is for a gene of DQA1_HUMAN.

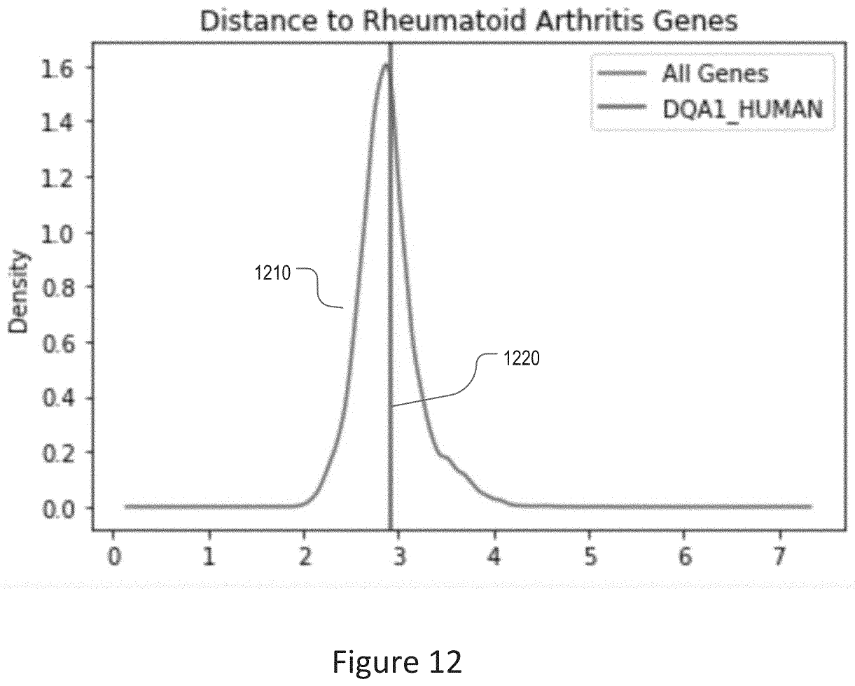

In addition, the distance 23 between a targeted gene and disease genes may also be utilized by the KnowGene model 16. A unit network distance may be defined as a path from one protein to another with a direct connection in the interactome. The shortest distances of a query gene to all the known genes of a given disease from 1 to 10 may be identified. Such distances may be binned. Each bin may be filed with the number of genes known to be associated with the given disease. For example, given a vector (n₁, n₂, . . . , n₁₀) with Σnᵢ=N_{g}, N_{g} may be the total number of known genes and nᵢ may be the number of genes in the known set with shortest distance i to the unknown gene. FIG. 12 illustrates an exemplary distribution of the distance to the disease genes. In FIG. 12, a density curve 1210 is for all genes, and a density curve 1220 is for a gene of DQA1_HUMAN.

For a given target, in an embodiment, the nearest neighbours 24 of targeted genes may be identified by calculating the cosine similarity of the target's embedding against all other embeddings in the dataset. This method may add another layer that provides an explanation of the association between a target and a disease, and may allow users to explore the embedding space. The result storage may include a platform that may be utilized to store the results and trained models created within the analytics pipeline.

In accordance with certain embodiments, embedding spaces may be generated from a knowledge graph. Often, machine learning on graphs may be limited in comparison with approaches used in vector spaces. Embeddings may be compressed representations of the data, which pack node properties in a vector, that are more practical to use in equation operations than an adjacency matrix that describes connections between nodes in a graph. Further, vector operations may be simpler and faster than comparable operations on graphs. Many embedding approaches are known in the art, including factorization approaches, random walk approaches, deep approaches, structural deep network embedding (SDNE) approaches, vertex embedding approaches, and graph embedding approaches. In an embodiment, the approach may comprise: sampling and relabeling sub-graphs around the selected node; training the model to maximize the probability of predicting a sub-graph that exists in the graph on the input; and computing embedding spaces based on a hidden layer. Accordingly, technical improvements are realized when a computing device structures information into embedding spaces based on knowledge graphs and runs search queries on the embedding spaces, which specifically result in the retrieval of more relevant and accurate information, in a shorter amount of time. Furthermore, calculations may be performed to predict the relationship between a gene target and a disease, and rank the predictions using scoring functions.

In some embodiments, the disclosed systematic data integration and curation may: facilitate target rationale reviews (TADR); refine therapeutic hypotheses; and prioritize best emerging targets/pathways that may serve as a basis for follow-up target validation. Such analyses may integrate human genetics, functional genomics, immunophenotyping, network analysis, and curation (e.g., disease pathobiology, gene function, existing/failed drugs, competitive landscape, internal and external sources). In an embodiment, this disclosure may provide a framework that: may integrate key datasets and supports key analytical methods to prioritize targets/pathways; may be searchable by any combination of filters; may be a scalable, sustainable solution; and may be flexible and evolutive, to enable future integration of new data (increased data size and new datatypes) and supports new analytics with the ability to identify newly available data sets, upload, process and summarize them for internal review.

In some embodiments, this disclosure may assist a discovery scientist to answer key scientific questions for a new drug target before going into clinical trial. The more informed and prioritized targets will not only result into better drugs, but also better selection of patients. This disclosure may address key scientific topics, in certain embodiments, including without limitation: the therapeutic landscape of a certain disease-target combination; known disease biological data for the targeted gene; human genetics evidence for the targeted gene; and/or, differential expression datasets available for the target of interest. These topics may assist with the selection of the patient population that may be selected for clinical trials. The present disclosure may provide a novel framework for identifying new drug targets. In some embodiments, the system may include a defined schema for connecting different data sources that may be employed in various analytics methods. This may include the use of knowledge graph, and weighted edges in the knowledge graphs for discovering new links. These weights may influence the prediction scores of targets.

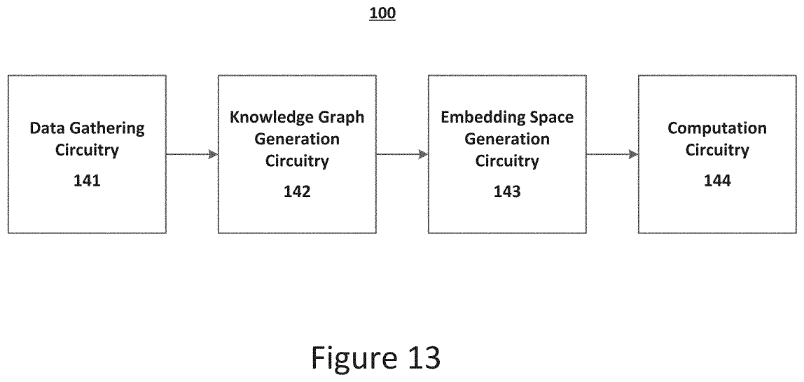

In an embodiment, as shown in FIG. 13, a data gathering circuitry 141 may be configured to receive human data, such as genetic information and disease information. A knowledge graph generation circuitry 142 may be configured to generate a graphically represented data structure model based on the received dataset. The knowledge graph generation circuitry 142 may construct a knowledge graph from the received information that is mapped to a predefined graph schema, in accordance with certain embodiments. The resulting graphical representation may provide a specific format of structured data where each connecting edge represents a relationship between nodes. The knowledge graph generation circuitry 142 may further train the knowledge graph, in accordance with certain embodiments.

The system 100 may further include an embedding space generation circuitry 143 that may be configured to generate embedding spaces based on knowledge graphs. The embedding space generation circuitry 143 may convert the data and relationships represented in the knowledge graph into a plot of nodes within an embedding space. The generated embedding space may include vector nodes (e.g., vector set of triplets) representing the structured information included in the knowledge graph.

In some embodiments, the system 100 may include a computation circuitry 144 for implementing computations within the embedding space. For example, the computation circuitry 144 may be configured to: determine a plurality of candidate statements; determine a weighting from the combination index (CI) database based on the query; determine a score for each candidate statement based on the query and based on the weighting using the embedding space for the knowledge graph; analytics modeling; and/or, rank the predicted links between the target and a disease. The computation circuitry 144 may enable the modeling of weighting in order to score the prediction of the relationship between a target and a disease. In addition, the computation circuitry 144 may identify gap regions within the region of interest, and compute Max-Min Multi-dimensional computations to determine a center for the gap regions within the region of interest. The computation circuitry 144 is further configured to consider that center node to be an embedding of a newly discovered gene target that was not present in the original knowledge graph. This may be technically implemented by generating a new node within the embedding space at the determined center having the attributes of the newly discovered target. Overall, executing the scoring process provides improvements to the computing capabilities of a computer device executing the process by reducing the search space and by allowing for more efficient data analysis to analyze large amounts of data in a shorter amount of time.

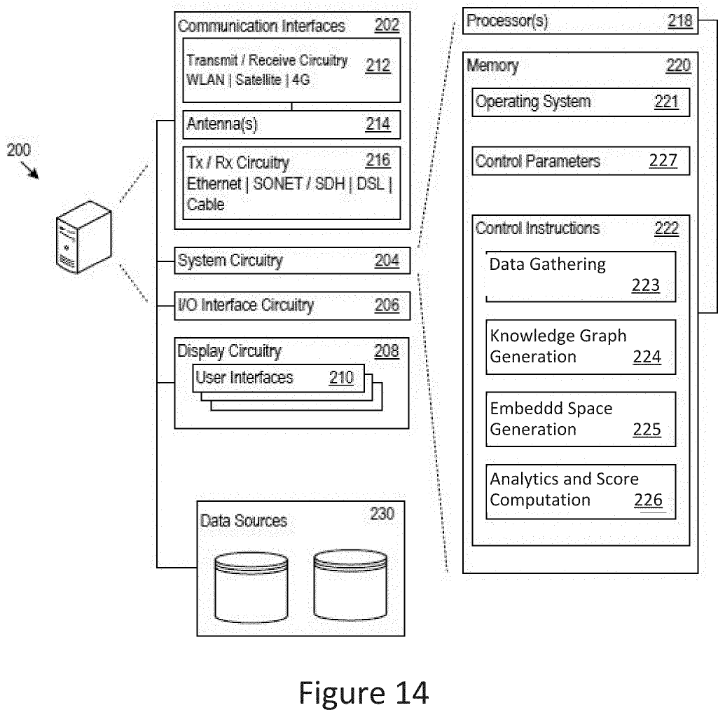

FIG. 14 illustrates a computer architecture of a computer device 200 on which the features of the system 100 may be executed. The computer device 200 includes communication interfaces 202, system circuitry 204, input/output (I/O) interface circuitry 206, and display circuitry 208. The graphical user interfaces (GUIs) 210 displayed by the display circuitry 208 may be representative of GUIs generated by the system 100 to present a query to an enterprise application or end user, requesting information on a compound to be replace and/or compound attributes desired to be satisfied by a candidate discovery compound. The graphical user interfaces (GUIs) 210 displayed by the display circuitry 208 may also be representative of GUIs generated by the system 100 to receive query inputs identifying the compound to be replace and/or compound attributes desired to be satisfied by a candidate discovery compound. The GUIs 210 may be displayed locally using the display circuitry 208, or for remote visualization, e.g., as HTML, JavaScript, audio, and video output for a web browser running on a local or remote machine. Among other interface features, the GUIs 210 may further render displays of any new formulations resulting from the replacement of compounds(s) with discovery compound(s) selected from the processes described herein.

The GUIs 210 and the I/O interface circuitry 206 may include touch sensitive displays, voice or facial recognition inputs, buttons, switches, speakers and other user interface elements. Additional examples of the I/O interface circuitry 206 includes microphones, video and still image cameras, headset and microphone input/output jacks, Universal Serial Bus (USB) connectors, memory card slots, and other types of inputs. The I/O interface circuitry 206 may further include magnetic or optical media interfaces (e.g., a CDROM or DVD drive), serial and parallel bus interfaces, and keyboard and mouse interfaces.

The communication interfaces 202 may include wireless transmitters and receivers (herein, “transceivers”) 212 and any antennas 214 used by the transmit-and-receive circuitry of the transceivers 212. The transceivers 212 and antennas 214 may support WiFi network communications, for instance, under any version of IEEE 802.11, e.g., 802.11n or 802.11ac, or other wireless protocols such as Bluetooth, Wi-Fi, WLAN, cellular (4G, LTE/A). The communication interfaces 202 may also include serial interfaces, such as universal serial bus (USB), serial ATA, IEEE 1394, lighting port, I²C, slimBus, or other serial interfaces. The communication interfaces 202 may also include wireline transceivers 216 to support wired communication protocols. The wireline transceivers 216 may provide physical layer interfaces for any of a wide range of communication protocols, such as any type of Ethernet, Gigabit Ethernet, optical networking protocols, data over cable service interface specification (DOCSIS), digital subscriber line (DSL), Synchronous Optical Network (SONET), or other protocol.

The system circuitry 204 may include any combination of hardware, software, firmware, APIs, and/or other circuitry. The system circuitry 204 may be implemented, for example, with one or more systems on a chip (SoC), application specific integrated circuits (ASIC), microprocessors, discrete analog and digital circuits, and other circuitry. The system circuitry 204 may implement any desired functionality of the system 100. As just one example, the system circuitry 204 may include one or more instruction processor 218 and memory 220.

The memory 220 stores, for example, control instructions 222 for executing the features of the system 100, as well as an operating system 221. In one implementation, the processor 218 executes the control instructions 222 and the operating system 221 to carry out any desired functionality for the scoring system 100, including those attributed to data gathering 223 (e.g., relating to the data gathering circuitry 141), knowledge graph generation 224 (e.g., relating to the knowledge graph generation circuitry 142), embedding space generation 225 (e.g., relating to the embedding space generation circuitry 143), and/or analytics/score computation 226 (e.g., relating to the computation circuitry 144). The control parameters 227 provide and specify configuration and operating options for the control instructions 222, operating system 221, and other functionality of the computer device 200.

The computer device 200 may further include various data sources 230. Each of the databases that are included in the data sources 230 may be accessed by the system 100 to obtain data for consideration during any one or more of the processes described herein. For example, the data gathering circuitry 141 may access the data sources 230 to obtain the information for generating the knowledge graph and the embedding space.

While the present disclosure has been particularly shown and described with reference to an embodiment thereof, it will be understood by those skilled in the art that various changes in form and details may be made therein without departing from the spirit and scope of the present disclosure. Although some of the drawings illustrate a number of operations in a particular order, operations that are not order-dependent may be reordered and other operations may be combined or broken out. While some reordering or other groupings are specifically mentioned, others will be apparent to those of ordinary skill in the art and so do not present an exhaustive list of alternatives.

What is claimed is:

1. A system for identifying a target gene associated with a disease, comprising:
a memory to store executable instructions; and
a processor adapted to access the memory, the processor further adapted to execute the executable instructions stored in the memory to:
extract datasets from a plurality of databases, the extracted datasets comprising historical datasets for a genetic mutation in a human DNA dataset, and/or a gene expression dataset, and/or a gene interaction dataset, and/or a drug dataset, and/or a disease dataset,
store the extracted datasets in a data lake, the data lake stored in the memory, the data lake stored in graph-based datasets, each of the graph-based datasets comprising a subject and an object and a predicate, and
generate a knowledge graph based on the data lake, the knowledge graph representing a plurality of links related to at least one gene and at least one disease.

2. The system of claim 1, wherein the processor is further adapted to:
display the knowledge graph.

3. The system of claim 1, wherein the processor is further adapted to:
determine weighting scores for the plurality of links in the knowledge graph; and
predict a target link between a target gene and a target disease, the target link based on the weighting scores.

4. The system of claim 3, wherein the target link is predicted using a knowledge-graph based model for predicting gene-disease associations.

5. The system of claim 3, wherein the target link is predicted using an AmpliGraph model.

6. The system of claim 3, wherein:
the knowledge graph includes numerical values as weights, associated with at least a portion of the plurality of links represented in the knowledge graph; and
the target link is predicted using a link-prediction model based on the numerical values.

7. The system of claim 3, wherein the target link is predicted using at least one of a graph convolutional network model or a network based model including a KnowGene model.

8. A method for identifying a target gene associated with a disease, comprising the steps of:
extracting, by a device comprising a memory and a processor in communication with the memory, datasets from a plurality of databases, the extracted datasets comprising historical datasets for a genetic mutation in a human DNA dataset, and/or a gene expression dataset, and/or a gene interaction dataset, and/or a drug dataset, and/or a disease dataset;
storing, by the device, the extracted datasets in a data lake, the data lake stored in the memory, the data lake stored in graph-based datasets, each of the graph-based datasets comprising a subject and an object and a predicate; and
generating, by the device, a knowledge graph based on the data lake, the knowledge graph representing a plurality of links related to at least one gene and at least one disease.

9. The method of claim 8, further comprising the step of:
displaying, by the device, the knowledge graph.

10. The method of claim 8, further comprising the steps of:
determining, by the device, weighting scores for the plurality of links in the knowledge graph; and
predicting, by the device, a target link between a target gene and a target disease, the target link based on the weighting scores.

11. The method of claim 10, wherein the target link is predicted using a knowledge-graph based model for predicting gene-disease associations.

12. The method of claim 10, wherein the target link is predicted using an AmpliGraph model.

13. The method of claim 10, wherein:
the knowledge graph includes numerical values as weights associated with at least a portion of the plurality of links represented in the knowledge graph; and
the target link is predicted using a link-prediction model based on the numerical values.

14. The method of claim 10, wherein the target link is predicted using at least one of a graph convolutional network model or a network based model including a KnowGene model.

15. A non-transitory computer-readable medium including instructions configured to be executed by a processor, wherein the executed instructions are adapted to cause the processor to:
extract datasets from a plurality of databases, the extracted datasets comprising historical datasets for a genetic mutation in a human DNA dataset, and/or a gene expression dataset, and/or a gene interaction dataset, and/or a drug dataset, and/or a disease dataset;
store the extracted datasets in a data lake, the data lake stored in a memory in communication with the processor, the data lake stored in graph-based datasets, each of the graph-based datasets comprising a subject and an object and a predicate; and
generate a knowledge graph based on the data lake, the knowledge graph representing a plurality of links related to at least one gene and at least one disease.

16. The computer-readable medium of claim 15, wherein the executed instructions are further adapted to cause the processor to:
display the knowledge graph.

17. The computer-readable medium of claim 15, wherein the executed instructions are further adapted to cause the processor to:
determine weighting scores for the plurality of links in the knowledge graph; and
predict a target link between a target gene and a target disease, the target link based on the weighting scores.

18. The computer-readable medium of claim 17, wherein:
the target link is predicted using a knowledge-graph based model for predicting gene-disease associations; and
the target link is predicted using an AmpliGraph model.

19. The computer-readable medium of claim 17, wherein:
the knowledge graph includes numerical values as weights, associated with at least a portion of the plurality of links represented in the knowledge graph; and
the target link is predicted using a link-prediction model based on the numerical values.

20. The computer-readable medium of claim 17, wherein the target link is predicted using at least one of a graph convolutional network model or a network based model including a KnowGene model.
